(19) United States
(12) Patent Application Publication
(10) Pub. No.: US 2016/0089082 A1
(43) Pub. Date: Mar. 31, 2016

(54) Apparatus for Measuring Body Impedance and Method for Determining Use of Electrode Thereof

(71) Applicant: InBody Co., Ltd., Seoul (KR)

(72) Inventors: Ki Chul Cha, Seoul (KR); Kyung Ho Ryu, Seoul (KR)

(73) Assignee: InBody Co., Ltd., Seoul (KR)

(21) Appl. No.: 14/552,215

(22) Filed: Nov. 24, 2014

(30) Foreign Application Priority Data
Sep. 25, 2014 (KR) 10-2014-0128590

(51) Int. Cl. A61B 5/00; A61B 5/107; A61B 5/053

(52) U.S. Cl. CPC A61B 5/6843; A61B 5/0531; A61B 5/706; A61B 5/1072; A61B 5/7278; A61B 5/6829; A61B 5/6823; A61B 5/6824; A61B 5/6828; A61B 5/6825

(57) ABSTRACT

Disclosed is a body impedance measurement apparatus. The impedance measurement apparatus provides a plurality of electrodes for measuring body impedance, senses electrodes, contacting with a body, from among the plurality of electrodes, and determines the use of the electrodes contacting with the body, depending on the sensing result.

### CROSS-REFERENCE TO RELATED APPLICATIONS

A claim for priority under 35 U.S.C. §119 is made to Korean Patent Application No. 10-2014-0128590 filed Sep. 25, 2014, in the Korean Intellectual Property Office, the entire contents of which are hereby incorporated by reference.

### BACKGROUND

The inventive concepts described herein relate to a body impedance measuring apparatus and an operating method thereof.

A current electrode and a voltage electrode are necessary to measure body impedance. The current electrode is used to apply current to the human body, and the voltage electrode is used to measure a voltage of the human body. Conventionally, the body impedance is measured using touch-type electrodes contacting with the sole of the foot and touch-type electrodes contacting with a heel. This measurement apparatus is problematic in that the sole of the foot and the heel must exactly contact with the touch-type electrodes.

To solve the above-described problem, the Korea Publication No. 2008-0102581 discloses the shape of a footboard where a voltage electrode and a current electrode are alternately arranged. According to the Korea Publication No. 2008-0102581, the subject conveniently measures the body impedance without an effort to contact the sole of the foot and the heel with separate electrodes.

However, a voltage electrode and a current electrode need to be maximally spaced from each other to improve measurement precision about the body impedance. The Korea Publication No. 2008-0102581 discloses a footboard where a voltage electrode and a current electrode are alternately arranged. In this case, the separation between the voltage electrode and the current electrode makes it difficult to improve precision. In addition, electrodes disclosed in the Korea Publication No. 2008-0102581 are not changed because the electrodes are classified into electrodes for voltage and electrode for current, thereby making it impossible to conduct measurement appropriate to the subject's body condition. The U.S. Publication No. 2005-0228449 and the U.S. Pat. No. 6,850,798 disclose a technique for measuring the body impedance of a person or an animal using a plurality of electrodes; however, electrodes are not switched to measure the body impedance of the subject exactly regardless of a posture of a measurer.

Accordingly, there is required a body impedance measurement apparatus capable of changing electrode characteristics to be appropriate to the subject's body condition and adjusting a movable range of electrodes.

### SUMMARY

According to one aspect, a body impedance measurement apparatus provides a plurality of electrodes for measuring body impedance and determines the use of electrodes, contacting with a body, from among the plurality of electrodes.

The body impedance measurement apparatus senses electrodes, contacting with a body, from among the plurality of electrodes and determines the use of electrodes contacting with the body depending on the sensing result.

The body impedance measurement apparatus includes a switch which is disposed between the plurality of electrodes and an impedance measurement circuit. The switch switches electrodes contacting with a body to current electrodes or voltage electrodes or sets the electrodes to an open state. The use of electrodes is determined depending on an operation of the switch.

The body contacting with an electrode is the sole of the foot, a palm, an ankle, a waist, a trunk, a forearm, or a thigh, and electrodes of which the use is determined are electrodes, contacting with the sole of the foot, the palm, the ankle, the waist, the trunk, the forearm, or the thigh, from among the plurality of electrodes.

According to another aspect, an operating method of a body impedance measurement apparatus includes sensing electrodes, contacting with a body, from among a plurality of electrodes provided to measure body impedance; and determining the use of the electrodes contacting with the body, depending on the sensing result.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a diagram showing a footboard and electrodes of a body impedance measurement apparatus according to an embodiment of the inventive concept;

FIG. 2 is a diagram showing a shape of a pad for measuring body impedance of a trunk, an arm, and a leg;

FIG. 3 is a block diagram schematically illustrating a body impedance measurement apparatus according to an embodiment of the inventive concept;

FIG. 4 is a diagram showing arrangements of electrodes according to an embodiment of the inventive concept;

FIG. 5 is a diagram showing another embodiment of arrangement of electrodes; and

FIG. 6 is a flow chart showing an operating method of a body impedance measurement apparatus according to an embodiment of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

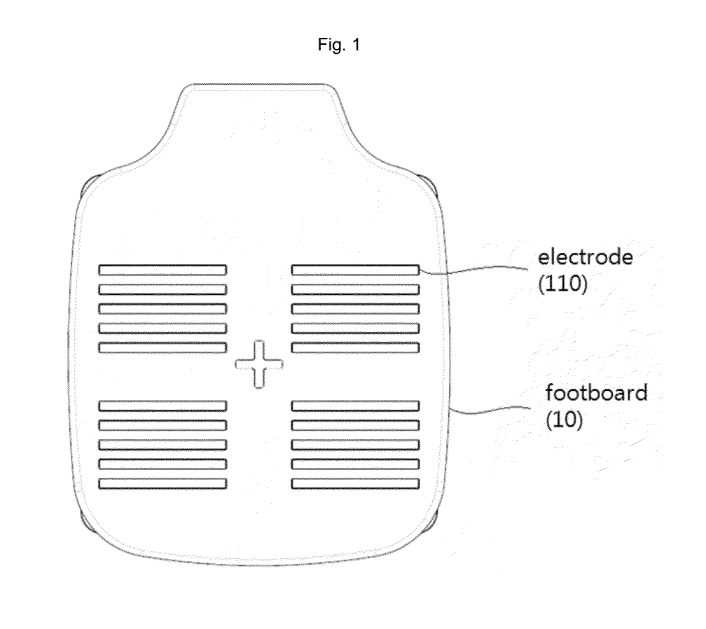

FIG. 1 is a diagram showing a footboard and electrodes of a body impedance measurement apparatus according to an embodiment of the inventive concept.

Referring to FIG. 1, a body impedance measurement apparatus according to an embodiment of the inventive concept includes a footboard 10 and electrodes 110. Although not shown in FIG. 1, the body impedance measurement apparatus according to an embodiment of the inventive concept may further contain the following generally needed to measure body impedance: a display unit, an operation unit, an upper body, and a supporting member.

The body impedance measurement apparatus contains the plurality of electrodes 110 for measurement of the body impedance and determines the use of electrodes, contacting with the body, from among the plurality of electrodes 110. The body impedance measurement apparatus senses the plurality of electrodes 110 and determines the use of electrodes contacting with the body depending on the sensing result.

The body contacting with electrodes 110 may be a part of the body, for example, the sole of the foot, palm, ankle, waist, trunk, forearm, or thigh. Electrodes 110 of which the use is determined may be electrodes 110, sensed as contacting with the sole of the foot, palm, ankle, waist, trunk, forearm, or thigh, from among the plurality of electrodes 110.

The body impedance measurement apparatus senses electrodes 110 contacting with the body to measure a length of the body. The body impedance measurement apparatus measures the length of the body, based on the number of electrodes 110 contacting with the body and distances among the electrodes 110. The body impedance measurement apparatus determines the use of electrodes 110 depending on an operation of a switch.

The body impedance measurement apparatus senses electrodes contacting with the body to measure a length of the body. The body impedance measurement apparatus measures the length of the body, based on the number of electrodes contacting with the body and distances among the electrodes. The body impedance measurement apparatus measures the length of the body depending further on body impedance.

The footboard 10 contacts with both feet of the subject and is configured such that the subject steps onto the footboard 10. In FIG. 1, an embodiment of the inventive concept is exemplified as the footboard 10 has a tetragonal shape. However, the shape of the footboard 10 may be variously modified or changed such that the subject steps onto the footboard 10. Arrangement of the electrodes 110 shown in FIG. 1 is only illustrative, and the electrodes 110 may be arranged in various manners.

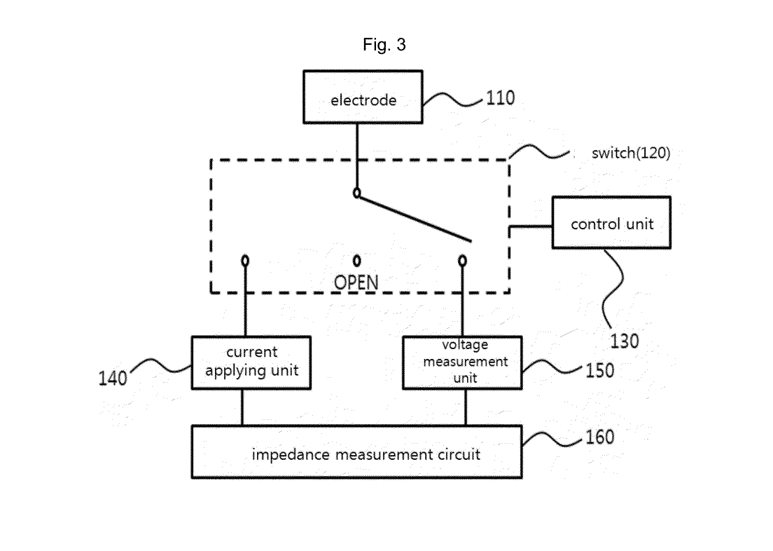

The electrodes 110 may be arranged on at least a part of an upper surface of the footboard 10. The electrodes 110 may be disposed on the upper surface of the footboard to have a variety of shapes. The electrodes 110 may be disposed on the whole upper surface of the footboard 10. A structure and an operation of the electrodes 110 will be more fully described with reference to FIG. 3.

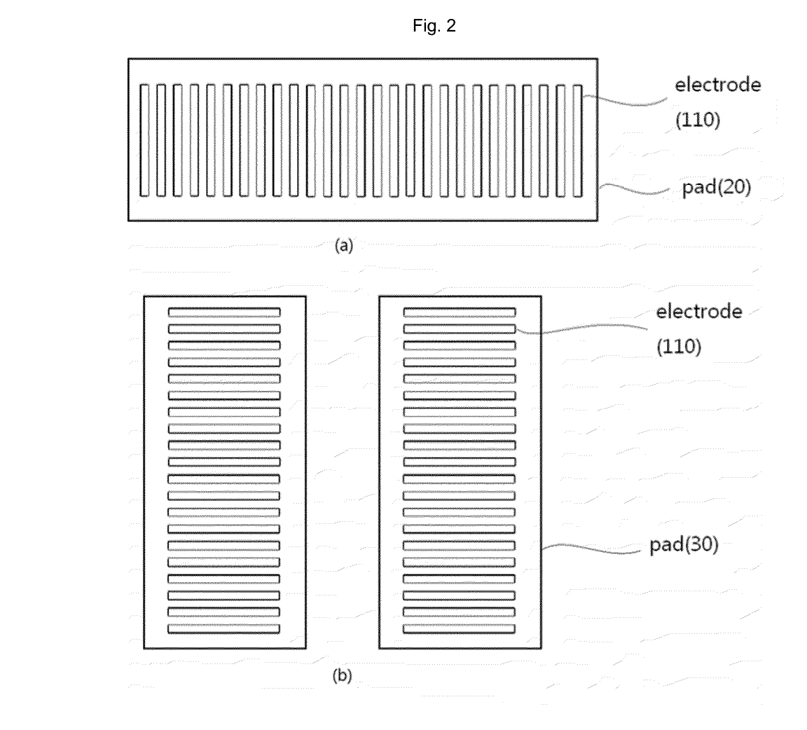

The body impedance measurement apparatus may include a pad, adapted to measure various body parts of the subject, and a supporting member as well as the footboard 10. For example, the body impedance measurement apparatus may contain various pads that allow contacts with hands, foots, abdomen, and so on. Various pad shapes will be more fully described with reference to FIG. 2.

FIG. 2 is a diagram showing a shape of a pad for measuring body impedance of a trunk, an arm, and a leg.

Referring to FIG. 2, there are illustrated a pad 20 for measuring trunk's body impedance, a pad 30 for measuring arm's or leg's body impedance, and electrodes 110 disposed on the pads 20 and 30.

The pad 20 has a shape allowing a contact with the trunk. The pad 20 may have various lengths and widths. The pad 20 may have a width and a length allowing a contact with the trunk of the subject.

The pad 20 is configured to have a plane or curved shape enabling an easy contact with the trunk of the subject. The pad 20 may also be flexible to easily contact with the trunk of the subject.

The pad 20 contains a plurality of electrodes 110. A body impedance measurement apparatus senses electrodes 110 that contacts with the trunk of the subject. The body impedance measurement apparatus determines the use of the electrodes 110, contacting with the trunk of the subject, depending on the sensing result.

The body impedance measurement apparatus senses electrodes 110 contacting with the subject's trunk to measure the length or area of the trunk. The body impedance measurement apparatus may measure the length or area of the trunk depending on the number of electrodes 110 contacting with the subject's trunk and distances among the electrodes 110.

The pad 30 has a shape allowing a contact with the leg or arm. The pad 30 may have various lengths and widths. The pad 30 may have a width and a length allowing a contact with the subject's leg or arm. The pad 30 is formed of a pair.

The pad 30 is configured to have a plane or curved shape enabling an easy contact with the subject's leg or arm. The pad 30 may also be flexible easily to contact with the subject's leg or arm.

The pad 30 contains a plurality of electrodes 110. The body impedance measurement apparatus senses electrodes 110 that contacts with the subject's leg or arm. The body impedance measurement apparatus determines the use of the electrodes 110, contacting with the subject's leg or arm, depending on the sensing result.

The body impedance measurement apparatus senses electrodes 110 contacting with the subject's leg or arm to measure the length or area of the leg or arm. The body impedance measurement apparatus may measure the length or area of the leg or arm depending on the number of electrodes 110 contacting with the subject's leg or arm and distances among the electrodes 110.

FIG. 3 is a block diagram schematically illustrating a body impedance measurement apparatus according to an embodiment of the inventive concept.

Referring to FIG. 3, a body impedance measurement apparatus contains an electrode 110 and a switch 120. The body impedance measurement apparatus further includes a control unit 130, a current applying unit 140, a voltage measurement unit 150, and an impedance measurement circuit 160.

The electrode 110 may be exposed on a footboard 10 or pads 20 and 30 for measurement of body impedance. The electrode 110 may be a reconfigurable electrode. That is, the electrode may be an electrode of which the characteristic is not defined: a voltage electrode for measuring a voltage of the subject or a current electrode for applying current to the subject. The electrode 110 is a conductor contacting with the body of the subject, and a characteristic of the electrode 110 is selected by the switch 120.

The switch 120 selects a characteristic of the electrode 110. That is, the switch 120 switches the electrode 110 to a current electrode or a voltage electrode. Also, the switch 120 sets the electrode 120 to an open state.

The switch 120 connects the electrode 110 to the current applying unit 140 to use the electrode 110 as the current electrode. Also, the switch 120 connects the electrode 110 to the voltage measurement unit 150 to use the electrode 110 as the voltage electrode. The switch 120 is disposed between the electrode 110 and the impedance measurement circuit 160.

The current applying unit 140 applies current to a part of the subject's body, contacting with the footboard 10 or the pads 20 and 30, through the electrode 110. Although not shown in figure, the current applying unit 140 may be connected to a voltage supplying unit for applying current to the subject. The amount of current applied may be decided by the control unit 130.

The voltage measurement unit 150 measures a voltage of the subject through the electrode 110. When contacting with the electrode 110, the subject's body acts as a resistor, thereby making it possible for the voltage measurement unit 150 to measure the subject's voltage by means of the current applied by the current applying unit 140. The subject's voltage measured by the voltage measurement unit 150 is transferred to the impedance measurement circuit 160.

The impedance measurement circuit 160 measures body impedance of the subject. The impedance measurement circuit 160 measures the body impedance of the subject, depending on the amount of current applied by the current applying unit 140 and a level of a voltage measured by the voltage measurement unit 150. The impedance measurement circuit 160 may conduct impedance measurement about the subject's body every body part. Although specially disclosed in this specification, the impedance measurement circuit 160 may perform a generally known body impedance measurement operation.

The control unit 130 controls the switch 120.

The control unit 130 connects the switch 120 to the current applying unit 140 such that the switch 120 acts as a current electrode. The control unit 130 connects the switch 120 to the voltage measurement unit 150 such that the switch 120 acts as a voltage electrode. The control unit 130 makes the switch 120 open.

To measure the body impedance, the control unit 130 senses the electrode 110 contacting with the subject's body, depending on a position of the subject's body put on the footboard 10 or the pads 20 and 30. The subject's body may be a leg, an abdomen, a leg, an arm, and so on. The control unit 130 controls the switch 120 depending on the sensing result such that the switch 120 switches the electrode 110 to a current electrode or a voltage electrode or remains open.

For example, the control unit 130 controls the switch 120 so as to switch the electrode 110, contacting with the subject's body, to the current electrode or the voltage electrode or such that the electrode 110 not contacting with the subject's body is set to an open state.

As described above, the body impedance measurement apparatus according to an embodiment of the inventive concept reconfigures the electrode 110 such that a voltage electrode and a current electrode are sufficiently spaced from each other, thereby improving measurement precision about the body impedance. Further, the body impedance measurement apparatus according to an embodiment of the inventive concept switches the electrode 110 according to the foot's size to measure the body impedance conveniently without the subject adjusting a foot position. In addition, the body impedance measurement apparatus according to an embodiment of the inventive concept precisely measures the body impedance regardless of the foot's size. The body impedance measurement apparatus according to an embodiment of the inventive concept makes non-contacted electrodes open, thereby improving measurement precision about the body impedance and efficiency of a power consumed to measure the body impedance.

Under a control of the control unit 130, electrodes 110, placed at a first region, from among electrodes 110 contacting with the subject's body is switched to current electrodes, and electrodes 110, placed at a second region, from among the electrodes 110 contacting with the subject's body is switched to voltage electrodes. In exemplary embodiments, the first region and the second region are opposite to each other within a region contacting with the subject's body. When the subject's body is a foot, for example, the first region that includes electrodes 110 sensed as contacting with the foot is a part of the front (e.g., the vicinity of toe) of the foot, and the second region that includes electrodes 110 sensed as contacting with the foot is a part of the rear (e.g., the vicinity of heel) of the foot.

According to an aspect of the inventive concept, the electrodes 110 according to an embodiment of the inventive concept may be disposed on the footboard 10 together with general electrodes. In general, current electrodes are assigned to the front of the foot, and voltage electrodes are assigned to the rear of the foot. Thus, current electrodes are disposed at the front of the footboard 10, voltage electrodes at the rear of the footboard 10, and the electrodes 110 at the center of the footboard 10.

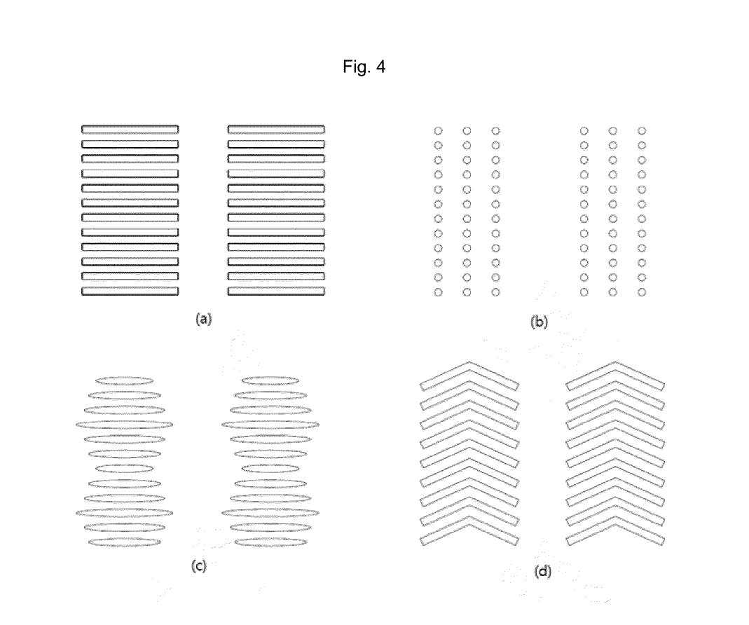

FIG. 4 is a diagram showing arrangements of electrodes according to an embodiment of the inventive concept.

Referring to FIG. 4, electrodes are arranged in various shapes. Electrodes 110 according to an embodiment of the inventive concept may be arranged in various other shapes as well as in shapes corresponding to (a) through (d) of FIG. 4. The electrode arrangement shown in FIG. 4 is only exemplary. Arrangement of electrodes according to embodiments of the inventive concept is not limited to that shown in FIG. 4, and the scope and spirit of the inventive concept is not interpreted as being restrictive by the electrode arrangement shown in FIG. 4.

The electrodes 110 may be disposed to be sufficiently wide considering the foot's size. Even though the electrodes 110 are widely disposed in consideration of the foot's size of the subject, the electrodes 110 act as voltage electrodes or current electrodes only in a region contacting with the subject's body, thereby making it possible to measure the body impedance precisely regardless of the body part of the subject.

Also, electrodes 110, placed at the front, from among the electrodes 110 may act as current electrodes, and electrodes 110, placed at the rear, from among the electrodes 110 may act as voltage electrodes. Positions of the front and rear may be decided considering the foot's size of the subject.

The foot's size of the subject may be determined depending on a distribution of electrodes 110 sensed as contacting with the foot. The area where there are disposed electrodes 110 that will act as voltage electrodes or current electrodes may be determined depending on measurement precision about the body impedance. The electrodes 110 may be variously arranged to include a shape of the sole of the foot.

The electrodes 110 may be disposed on the whole upper surface of a footboard 10. Now that the electrodes 110 according to an embodiment of the inventive concept allows measuring the subject's body impedance regardless of the size or position of the subject's foot, electrodes 110 corresponding to positions of both feet may have a symmetrical shape or may not have specific arrangement.

Activated are electrodes, sensed as contacting with the subject's body, from among the electrodes 110 disposed on the whole surface of the footboard 10. A part of the activated electrodes may be used as voltage electrodes, and the rest thereof may be used as current electrodes. Even though disposed on the whole supper surface of the footboard 10 or a pad 20 or 30, the electrodes 110 according to an embodiment of the inventive concept recognize the size and position of the body part of the subject, thereby making it possible to measure the subject's body impedance precisely.

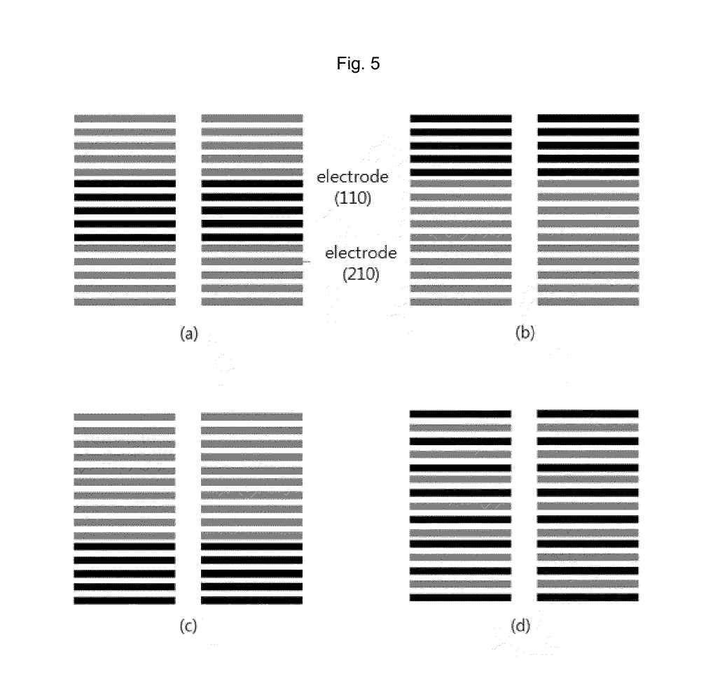

FIG. 5 is a diagram showing another embodiment of arrangement of electrodes.

Referring to FIG. 5, electrodes 110 according to an embodiment of the inventive concept and general electrodes 210 are disposed together. The general electrodes 210 may be general voltage or current electrodes. An embodiment of the inventive concept is exemplified as the electrodes 110 according to an embodiment of the inventive concept and the general electrodes 210 are disposed together. Arrangement of electrodes according to embodiments of the inventive concept is not limited to that shown in FIG. 5, and the scope and spirit of the inventive concept is not interpreted as being restrictive by the electrode arrangement shown in FIG. 5.

Referring to a (a) case of FIG. 5, the electrodes 110 are disposed at the center, and the electrodes 210 are disposed at the front and rear. According to an aspect of the inventive concept, the electrodes 110 are disposed at the center, current electrodes at the front, and voltage currents at the rear. According to another aspect of the inventive concept, the electrodes 110 are disposed at the center, the voltages electrodes at the front, and the current currents at the rear. Referring to (b) through (d) cases of FIG. 5, the electrodes 110 and the electrodes 210 are disposed together at various positions and in various ratios.

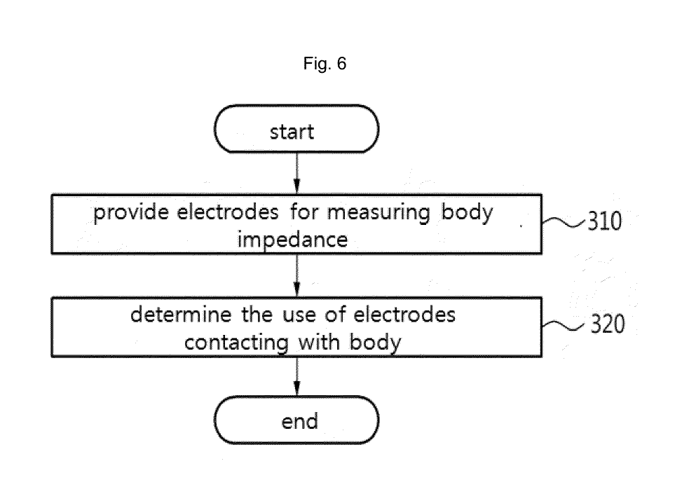

FIG. 6 is a flow chart showing an operating method of a body impedance measurement apparatus according to an embodiment of the inventive concept.

Referring to FIG. 6, in step S310, a body impedance measurement apparatus provides a plurality of electrodes for measuring body impedance.

In step S320, the body impedance measurement apparatus determines the use of electrodes 110, contacting with the body, from among the plurality of electrodes 110. The body impedance measurement apparatus may sense electrodes, contacting with the body, from among the plurality of electrodes 110 provided to measure the body impedance. The body contacting with electrodes may be the sole of the foot, palm, ankle, waist, trunk, forearm, or thigh, and electrodes of which the use is determined may be electrodes sensed as contacting with the sole of the foot, palm, ankle, waist, trunk, forearm, or thigh. The body impedance measurement apparatus may determine the use of the electrodes 110 depending on the sensing result.

The body impedance measurement apparatus switches a part of the plurality of electrodes 110 to current electrodes or voltage electrodes or sets the electrodes 110 to an open state. A switch that switches an electrode contacting with the body to a current electrode or a voltage electrode or makes an electrode open may be disposed between the electrode and an impedance measurement circuit. The body impedance measurement apparatus may determine the use of the electrode depending on an operation of the switch.

The body impedance measurement apparatus senses an electrode contacting with the body to measure the length of the body. Also, the body impedance measurement apparatus may measure the length of the body depending on the number of electrodes contacting with the body and distances among the electrodes. The body impedance measurement apparatus may measure the length of the body depending further on the body impedance.

Step S320 may include the step of switching electrodes, placed at a first region, from among electrodes contacting with a foot to current electrodes and electrodes, placed at a second region, from among the electrodes contacting with the foot to voltage electrodes. The first and second regions may be opposite to each other within a region contacting with the foot.

Step S320 may further include the step of opening electrodes not contacting with the foot. The body impedance measurement apparatus according to an embodiment of the inventive concept makes electrodes not contacting with the foot open, thereby improving efficiency of a power consumed to measure the body impedance.

The body impedance measurement apparatus according to an embodiment of the inventive concept can precisely measure the subject's body impedance regardless of the size or position of the subject's foot through the above-described operation. In the body impedance measurement apparatus according to an embodiment of the inventive concept, a current-applied position and a voltage-measured position are sufficiently spaced from each other, thereby improving measurement precision about the body impedance. The body impedance measurement apparatus according to an embodiment of the inventive concept can precisely measure the body impedance regardless of areas of various body parts. The body impedance measurement apparatus according to an embodiment of the inventive concept can improve measurement precision of body impedance by applying current to an appropriate position and measuring a voltage.

The example embodiments may be recorded in non-transitory computer-readable media including program instructions to perform various operations embodied by a computer. The media may also include, alone or in combination with the program instructions, data files, data structures, and the like. The media and program instructions may be those specially designed and constructed for the purposes, or they may be of the kind well-known and available to those having skill in the computer software arts. Examples of non-transitory computer-readable media include magnetic media such as hard disks, floppy disks, and magnetic tape; optical media such as CD ROM disks and DVD; magneto-optical media such as floptical disks; and hardware devices that are specially to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be to act as one or more software modules in order to perform the operations of the above-described embodiments.

While the inventive concept has been described with reference to exemplary embodiments, it will be apparent to those skilled in the art that various changes and modifications may be made without departing from the spirit and scope of the present invention. Therefore, it should be understood that the above embodiments are not limiting, but illustrative.

What is claimed is:

1. A body impedance measurement apparatus, comprising:
providing a plurality of electrodes for measuring body impedance; and
determining the use of electrodes, contacting with a body, from among the plurality of electrodes.

2. The body impedance measurement apparatus of claim 1, wherein the use of electrodes contacting with a body is determined depending on a result of sensing the electrodes, contacting with the body, from among the plurality of electrodes.

3. The body impedance measurement apparatus of claim 1, wherein a switch is disposed between the plurality of electrodes and an impedance measurement circuit,
wherein the switch switches electrodes contacting with a body to current electrodes or voltage electrodes or sets electrodes to an open state, and
wherein the use of electrodes is determined depending on an operation of the switch.

4. The body impedance measurement apparatus of claim 1, wherein a length of the body is measured depending on a result of sensing electrodes contacting with the body.

5. The body impedance measurement apparatus of claim 4, wherein the length of the body is measured depending on the number of electrodes contacting with the body and distances among the electrodes.

6. The body impedance measurement apparatus of claim 1, wherein a body contacting with an electrode is the sole of the foot, a palm, an ankle, a waist, a trunk, a forearm, or a thigh, and
wherein electrodes of which the use is determined are electrodes, contacting with the sole of the foot, the palm, the ankle, the waist, the trunk, the forearm, or the thigh, from among the plurality of electrodes.

7. The body impedance measurement apparatus of claim 2, further comprising:
a control unit adapted to control an operation of the switch.

8. The body impedance measurement apparatus of claim 7, wherein the control unit switches electrodes, placed at a first region, from among electrodes contacting with a foot to current electrodes and electrodes, placed at a second region, from among the electrodes contacting with the foot to voltage electrodes, and
wherein the first and second regions are opposite to each other within a region contacting with the foot.

9. The body impedance measurement apparatus of claim 1, further comprising:
a footboard contacting with each foot of the subject, and
wherein the plurality of electrodes are disposed on the whole upper surface of the footboard.

10. The body impedance measurement apparatus of claim 1, wherein the plurality of electrodes are disposed at a center of a footboard contacting with each foot of the subject, current electrodes are disposed at a front of the footboard, and voltage electrodes are disposed at a rear of the footboard.

11. An operating method of a body impedance measurement apparatus, comprising:
sensing electrodes, contacting with a body, from among a plurality of electrodes provided to measure body impedance; and
determining the use of the electrodes contacting with the body, depending on the sensing result.

12. The operating method of claim 11, further comprising:
sensing electrodes contacting with the body to measure a length of the body.

13. The operating method of claim 12, further comprising:
measuring a length of the body depending on the number of electrodes contacting with the body and distances among the electrodes.

14. The operating method of claim 13, wherein in measuring the length, the length of the body is measured depending further on body impedance.

15. The operating method of claim 12, wherein a body contacting with an electrode is a foot, and
wherein an electrode of which the use is an electrode, sensed as contacting with the foot, from among the plurality of electrodes.
